(19) United States
(12) Patent Application Publication
(10) Pub. No.: US 2017/0073627 A1
(43) Pub. Date: Mar. 16, 2017

(54) METHODS OF SELECTIVE CELL ATTACHMENT/DETACHMENT, CELL PATTERNIZATION AND CELL HARVESTING BY MEANS OF NEAR INFRARED RAYS

(71) Applicant: Industry-Academic Cooperation Foundation, Yonsei University, Seoul (KR)

(72) Inventors: Eun Kyung KIM, Seoul (KR); Hyun Ok Kim, Gyeonggi-do (KR); Jung Mok You, Seoul (KR); Jeong Hun Kim, Seoul (KR); Tea Hoon Park, Seoul (KR); Byeong Gwan Kim, Gyeonggi-do (KR); June Seok Heo, Seoul (KR); Han Soo Kim, Gyeonggi-do (KR)

(21) Appl. No.: 15/363,300

(22) Filed: Nov. 29, 2016

Related U.S. Application Data
(63) Continuation-in-part of application No. 14/342,451, filed on Mar. 3, 2014.

(30) Foreign Application Priority Data
Apr. 12, 2012 (KR) 10-2012-0037954

(51) Int. Cl. C12M 1/26; C08J 5/18; C12M 1/12; C12N 5/00; C12M 1/00

(52) U.S. Cl. CPC C12M 33/00; C12N 5/0068; C12M 23/20; C12M 25/06; C08J 5/18; C12N 2529/10; C12N 2533/30; C12N 2535/10; C12N 2539/10; C08J 2365/00; C08J 2333/14; C08J 2335/02; C08J 2333/16; C08J 2379/02

(57) ABSTRACT

The present invention relates to a method for selective cell attachment/detachment, cell patternization and cell harvesting by means of near infrared rays. More particularly, conducting polymers or metal oxides having exothermic characteristics upon irradiation of near infrared light is used as a cell culture scaffold, thus selectively attaching/detaching cells without an enzyme treatment. The scaffold has an effect of promoting proliferation or differentiation of stem cells, and therefore, can be used as a stem cell culture scaffold. The scaffold enables cell attachment/detachment without temporal or spatial restrictions, thus enabling cell patternization.

### CROSS-REFERENCE TO RELATED APPLICATION

This application is a continuation-in-part of co-pending U.S. application Ser. No. 14/342,451, filed Mar. 3, 2014.

### BACKGROUND

1. Field of the Invention

The present invention relates to methods for selectively detaching, patterning, and harvesting cells using near-infrared capable of being used in cell culture and detaching cells without trypsin.

2. Discussion of Related Art

Stem cells are cells having capabilities of self-replication and differentiation into at least two cells, and may be classified into totipotent stem cells, pluripotent stem cells, and multipotent stem cells.

Recently, therapeutic methods using such stem cells capable of being continuously self-replicated and differentiated into various tissues in the body are widely used, boosted by development of biotechnology. Particularly, such methods start to be used to treat incurable diseases such as Parkinson's disease, cancer, diabetes, etc. as well as human organ regeneration (Miyahara Y. et al., Nature Medicine, 12(4), 459-465, 2006; Kang, K. S. et al., Stem Cells, 24(6), 1620-1626, 2006; Silva, G. V. et al., Circulation, 18, 111, 2005). While various therapeutic methods using stem cells have been developed so far, there is still less research on the characteristics of stem cells, and there is a limit to treatment using stem cells due to limits to proliferation and differentiation of stem cells.

Generally, it is known that a fate of differentiated stem cells is often influenced by a cell to cell, and a cell to extracellular matrix (ECM) including growth factors, and also by an instructive environment (Nakayama et al, Neurosci Res, 46, 241-249, 2003). Recently, as research on interaction between an environment of stem cells and the stem cells, a bioengineering field is emerging. It is not a method of controlling a hormone, growth factor, or serum included in a cell culture, which is conventionally used in research or induction of the function of a cell, but a method of controlling attachment, proliferation, differentiation, and secretion to an extracellular matrix, which are characteristics of a cell, through interaction between a support to which the cell is attached and grown and the cell (Bauer S. et al., Acta Biomaterialia, 4, 1576-1582, 2008; Guo L. et al., Biomaterials, 29, 23-32, 2008). To this end, chemical surface modification which is used to develop a material having biocompatibility and change a surface characteristic is a critical factor.

The pluripotent stem cells can be differentiated into various cells and tissues derived from an ectoderm, a mesoderm, and an endoderm. These cells are derived from an inner cell mass located in a blastocyst generated after 4 to 5 days of fertilization, and called embryo stem cells. They are differentiated into various different tissues, but do not create a new organism.

The multipotent stem cells can be only differentiated into cells specific to tissues and organs in which these cells are included. They are involved in growth and development of tissues and organs in an embryonic period, a neonatal period, and an adult period, and functions of maintaining homeostasis of adult tissues and inducing regeneration of damaged tissues, and tissue-specific multipotent stem cells are generally called adult stem cells.

The adult stem cells are found in a stage in which individual organs of embryos are formed after development or at an adult stage, and differentiated only into cells generally constituting a specific tissue. Such adult stem cells serve to replenish the loss of cells normally or pathologically occurring in most of organs in an adult. Exemplary adult stem cells include hematopoietic stem cells (HSCs) and mesenchymal stem cells (MSCs). It is known that the HSCs are usually differentiated into blood cells in blood such as erythrocytes, leukocytes, and thrombocytes, and the MSCs are differentiated into cells of mesodermal tissues such as osteoblasts, chondroblasts, adipocytes, and myoblasts.

Stems cells can be differentiated into various cells according to how to differentiate or treat the stem cells. To control the differentiation capability of the stem cells, it is important to research and control the interaction between cell-to-cell and cell-to-extracellular matrix (ECM) including growth factors.

Generally, as a conventional technique to detach cells, an enzyme called trypsin is widely used. The trypsin chemically damages a bond in a cell attached to a cell culture container, resulting in damage to a cell wall or a protein present in the cell wall of a stem cell. Accordingly, when the trypsin is used, stem cells may be damaged, and thus degradation in proliferation capacity and differentiation potency may occur. In addition, since the trypsin is treated entirely to a culture container, it may be difficult to partially obtain a desired cell.

For this reason, there is a demand for developing a new technique to easily detach cells from a culture container, and to detach cells only from a desired part without damage to the cells.

### SUMMARY OF THE INVENTION

The present invention is directed to providing a cell culture container for culturing cells on a surface of a conductive compound or metal oxide film that can absorb near-infrared, and easily and selectively detaching cells without damage to the cells using a photothermal characteristic of the conductive compound or metal oxide by near-infrared radiation, a cell culture kit including the same, and a method of proliferating, differentiating, or detaching cells using the kit.

The present invention is also directed to providing a patterned substrate for cell culture for easily detaching cells using a photothermal characteristic of a conductive compound or metal oxide by near-infrared radiation.

One aspect of the present invention provides a cell culture container including a cell culture region in which a conductive polymer or metal oxide film having absorbance in a near-infrared region is formed.

Another aspect of the present invention provides a kit for cell culture including the cell culture container of the present invention and an apparatus for irradiating near-infrared.

Still another aspect of the present invention provides a method of proliferating or differentiating stem cells including culturing adult stem cells in a cell culture container.

Yet another aspect of the present invention provides a method of detaching cultured cells by irradiating a cell culture container with near-infrared.

Yet another aspect of the present invention provides a patterned substrate for cell culture, which includes a substrate and a cell culture region formed on the substrate and containing a conductive polymer or metal oxide film having an absorbance in a near-infrared region.

### BRIEF DESCRIPTION OF THE DRAWINGS

The above and other objects, features, and advantages of the present invention will become more apparent to those of ordinary skill in the art by describing in detail exemplary embodiments thereof with reference to the attached drawings, in which:

FIG. 1 is an absorption spectrum of a heterocyclic compound of Formula 1a according to the present invention;

FIG. 2 shows a photothermal effect by near-infrared absorption (808 nm) of a heterocyclic compound of Formula 1a according to the present invention;

FIG. 3 shows a proliferation rate of stem cells confirmed using an oxidized or reduced (reduced and thus neutral) film manufactured of a heterocyclic compound of Formula 1 d according to the present invention;

FIG. 4 is a microscope image showing detachment of stem cells cultured on a film manufactured of a heterocyclic compound of Formula 1a according to the present invention from a selective region by near-infrared irradiation;

FIG. 5 shows an a detached area of stem cells proportional to near-infrared irradiation time;

FIG. 6 is a microscope image of stem cells detached from a film manufactured of a heterocyclic compound of Formula 1e according to the present invention by near-infrared irradiation, and cultured in a new cell culture container; and

FIG. 7 shows results of differentiation of the stem cells detached from a film manufactured of a heterocyclic compound of Formula 1e according to the present invention by near-infrared irradiation into (a) osteocytes, (b) adipocytes, and (c) chondrocytes in a cell culture container for 16 days.

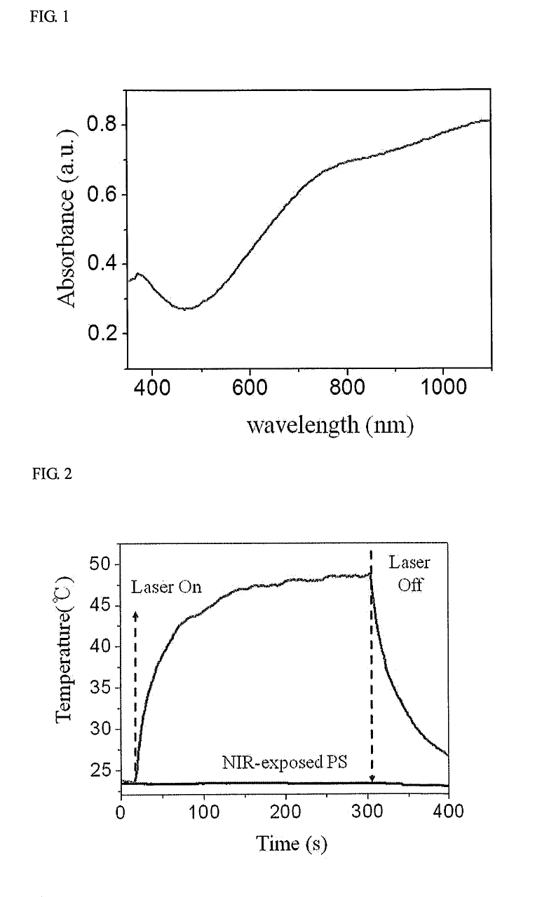

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of exemplary embodiments. The singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, components, and/or groups thereof, but do not preclude the presence or addition of one or more other features, integers, steps, operations, elements, components, and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

The present invention relates to a cell culture container including a cell culture region in which a conductive polymer or metal oxide film having an absorbance in a near-infrared region is formed.

The “cell culture container” used herein refers to a container used in conventional cell culture, and may be formed of a material suitable for cell culture, for example, any one of polycarbonate, polypropylene, polyethylene, polystyrene, polyurethane, polyethylene terephthalate, polyester, polyimide, polyethylene glycol, polydimethylsiloxane, or a copolymer or composite thereof; Nylon; paper; cotton; or glass. The material is preferably transparent to count cells under a microscope, but may be colored. The container may have a smooth surface, and may be formed in a round or square shape, but the present invention is not limited thereto. The container may be manufactured in a shape suitable for characteristics of a cell or its use, and by special treatment such as insertion of a regular pattern on a substrate. The cell culture container may be formed in a cylindrical, rectangular, or polygonal structure, but the present invention is not limited thereto. The cell culture container includes a cell culture region to which a cell is attached to be cultured, and may be a flask, or an enclosed structure such as a petri dish, but the present invention is not particularly limited thereto.

The cell culture container of the present invention is characterized by forming a conductive polymer or metal oxide film having an absorbance in a near-infrared region above a cell culture region in which cells are cultured in the cell culture container having the above-described shape and formed of the above-described material.

Since the conductive polymer or metal oxide film uses a photothermal characteristic of the conductive polymer or metal oxide generating heat by converting light energy into thermal energy due to absorption of near-infrared, when the film is used as a cell support, cells may be easily detached from the heat-generated part during the near-infrared irradiation without damage to a cell wall or a cell wall protein according to conventional trypsin treatment, and therefore repetitively used in cell culture and detachment.

In addition, according to one embodiment, when the film is used as a stem cell culture support, a proliferation rate of the stem cells are higher than that of stem cells in a common cell culture container, and the selectively detached stem cells can be subjected to additional culture and differentiation.

Accordingly, the conductive polymer or metal oxide film may be used as a support for cell proliferation or differentiation.

The conductive polymer or metal oxide film may be manufactured of a polymer or copolymer of a conductive monomer or a metal oxide having an absorbance in a near-infrared region.

In the present invention, near-infrared is in a wavelength range from 700 to 2500 nm, and conductive monomers having an absorbance in the near-infrared region of the present invention may also have an absorbance in the above range. According to an embodiment, in measurement of an absorbance at a wavelength of approximately 808 nm, when the near-infrared is irradiated for up to 300 seconds, a pyrogenic effect of approximately 25° C. may be exhibited.

The conductive monomer may be at least one selected from the group consisting of a heterocyclic compound represented by Formula 1 and aniline.

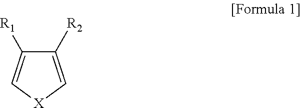

In Formula 1, X is N, O, S, Se, or Te,

R₁ and R₂ are the same as or different from each other, each of which is a hydrogen atom, —(CH₂)ₗ—O—(CH₂)m-(CF₂)ₙ—(CR₇R₈)ₖ—(CH₂)_{d}—Z,

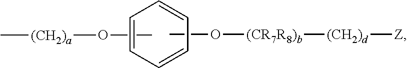

—O—CH(R₃)—CH(R₄)—O—, or —O—CH₂—C(R₅)(R₆)—CH₂—O—. However, R₁ and R₂ are not simultaneously hydrogen. R₃, R₄, R₅, and R₆ are the same as or different from each other, each of which is a hydrogen atom, —(CH₂)_{d}—Z, —(CH₂)ₗ—O—(CH₂)ₘ—(CF₂)ₙ—(CR₇R₈)ₖ—(CH₂)_{d}—Z, or

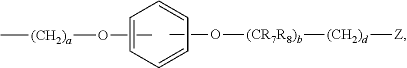

but when R₃ and R₄ are simultaneously hydrogen, R₅ and R₆ are not simultaneously hydrogen. R₇ and R₈ are the same as or different from each other, each of which is hydrogen, an alkyl group having 1 to 5 carbon atoms, or —(CH₂)_{d}—Z. Z is a methacrylate group or an acrylate group, l is an integer from 0 to 2, m is an integer from 0 to 3, n is an integer from 0 to 5, k is an integer from 0 to 4, a is an integer from 0 to 2, b is an integer from 0 to 7, and d is an integer from 0 to 2.

Preferably, the heterocyclic compound of Formula 1 may be at least one of Formulas 1a to 1k.

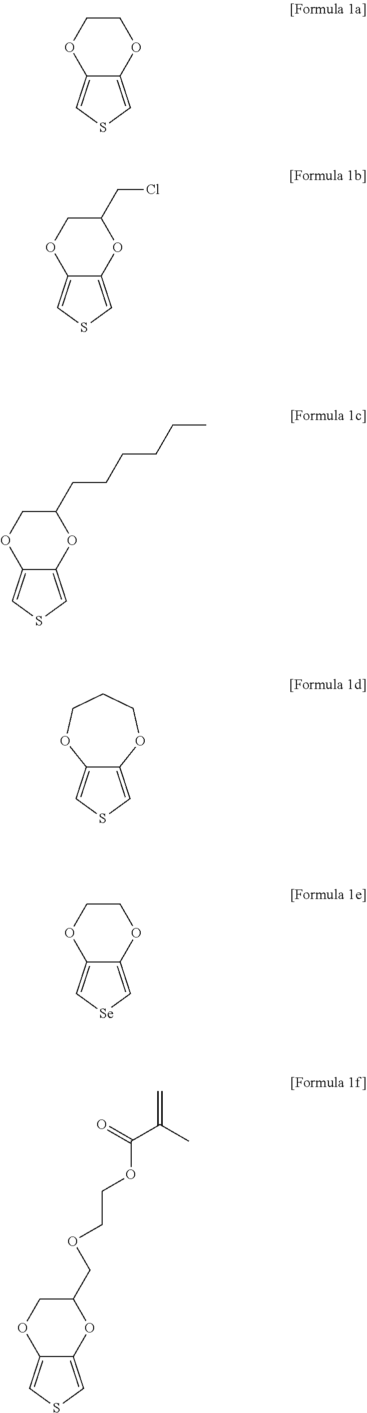 [SMILES: c1scc2c1OCCO2 ; ClCC1COc2cscc2O1 ; CCCCCCC1COc2cscc2O1 ; c1scc2c1OCCCO2 ; c1[se]cc2c1OCCO2 ; C=C(C)C(=O)OCCOCC1COc2cscc2O1]

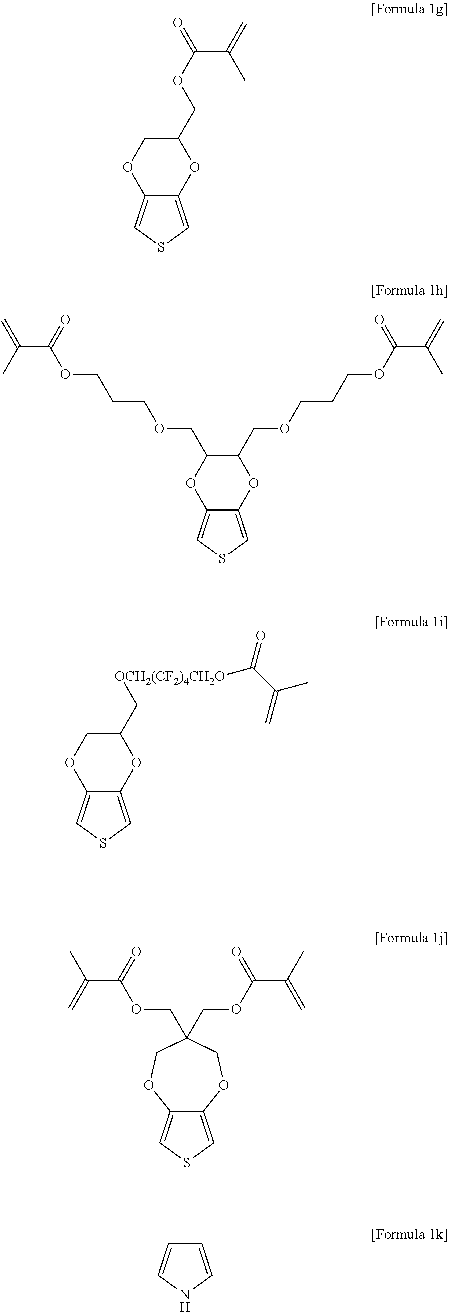

The conductive polymer may have a weight average molecular weight of 1,000 to 1,000,000 Da.

The conductive polymer refers to a polymerized product produced by polymerization of the above-described heterocyclic compound and/or aniline, which is a polymer or copolymer polymerized using an electrical, chemical, thermal, or optical method or an initiator.

The conductive polymer may be prepared by polymerizing a heterocyclic compound through solution polymerization using a conventional catalyst, electropolymerization using electricity [Macromolecular Research, 17, 791-796, 2009], vapor polymerization [Macromolecules, 43, 2322-2327, 2010], solution coating polymerization [Advanced Materials, 23, 4168-4173, 2011], or emulsion polymerization in an aqueous phase. The electropolymerization, vapor polymerization, solution coating polymerization, or emulsion polymerization for preparing particles used herein induces oxidative polymerization of the heterocyclic compound of the present invention, and the polymerization method using a conventionally used catalyst (acid, oxidant, etc.) is a conventional method used in polymerization of a monomer such as aniline as well as a heterocyclic compound.

In the method of preparing a conductive polymer film of the present invention, the conductive polymer may be directly coated on various substrates using the polymerization method, the conductive polymer dissolved in a solvent may be secondarily coated using various coating methods such as spin coating, printing coating, etc. after synthesis, a conductive polymer particle synthesized by an emulsion method may be dispersed in a solvent and secondarily coated to form a film. The present invention is not limited to the coating method, but various coating methods may be suitably used according to a compound or process, or the range of use or application range.

For example, when a doping state of a conductive polymer thin film is controlled, the conductive polymer thin film manufactured as described above is put into an electrolyte solution (solvent) without a monomer, circulated three times at a rate of 50 mV/s between 1 to −1 V using cyclic voltammetry, washed with a deionized solvent by removing power after the circulation is stopped for several seconds at a voltage (between 1 to −1 V) in a desired doping state, and dried.

The metal oxide may be magnesium oxide, strontium oxide, zinc oxide, aluminum oxide, or arsenic oxide, which is used alone or in combination of at least two thereof.

The conductive polymer or metal oxide film may have a thickness of 10 nm to 1 mm. When the thickness is less than 10 nm, the film is not easily formed and a photothermal phenomenon or an effect thereof occurring in the film is low. When the thickness of the film is more than 1 mm, it is difficult to form the film likewise, and when the absorbance of the material is high, it is necessary to transfer heat generated by the photothermal phenomenon from a part adjacent to a substrate, and here, time necessary to detach cells may be quite long. In addition, since the cell culture container of the present invention may be applied to a conventional cell culture, it is not limited to the kind of the cells, and for example, may be used in adult stem cell culture.

The “adult stem cells” used herein refer to stem cells shown in a stage in which an organ of an embryo is formed after development or an adult stage, and are only limited to cells generally differentiated into a specific tissue.

The adult stem cells of the present invention may be separated to use from adult stem cells derived from the breast, bone marrow, cord blood, blood, liver, skin, gastrointestine, placenta, or womb. The adult stem cells include neural stem cells capable of being differentiated into astrocytes, hematopoietic stem cells capable of being differentiated into myelocytes, mesenchymal stem cells capable of being differentiated into a bone, cartilage, lipid, muscle, etc., and liver stem cells capable of being differentiated into hepatocytes. Among these, the mesenchymal stem cells are cells having the ability of differentiation into various musculoskeletal cells such as osteocytes, chondrocytes, adipocytes, muscle cells, and fibrocytes.

Since the mesenchymal stem cells are present in cord blood (umbilical cord) and a bone marrow, they are more easily separated than other adult tissues, and there is an endeavor to use of the mesenchymal stem cells in treatment of various diseases including such musculoskeletal diseases. Unlike other stem cells, the mesenchymal stem cells are easily cultured to amplify in a bone marrow, unlike that has been known so far, the stem cells can be differentiated into mesoderm-, endoderm-, or ectoderm-derived cells, do not have rejection to immunity due to use of a self cell, and there is a bare chance that cells not differentiated in a desired direction, unlike embryonic stem cells, induce a cancer, which are very important in clinic.

The term “differentiation” used herein refers to a phenomenon in which a structure or function of cells is specified while the cells are divided, proliferated, and then developed, that is, a change in a shape or function of cells or tissues of an organism to execute a work given thereto. Generally, the differentiation is a phenomenon of dividing a system into at least two subsystems having different properties.

The term “proliferation” used herein refers an increase in the same kind of cells by division, that is, generally, an increase in cell counts in a multicellular organism. When a cell count reaches a certain level due to proliferation of cells, a trait (or characteristics) is generally differentiated and controlled. The increase in cells in the body and neogenesis of cytoplasms in cells are generally classified as growth. However, since the cell count increases in a biological aspect, it is appropriate that a period in which differentiation does not occur in an embryo stage of the multicellular organism is considered proliferation.

When adult stem cells are cultured on a conductive polymer or metal oxide film that is reduced and in a neutral state in the cell culture container of the present invention, proliferation of the cells increases, and when the cell culture container is irradiated with near-infrared, the cells are detached without damage due to a pyrogenic effect of the conductive polymer or metal oxide film, and the detached stem cells are transferred to a new cell culture container for normal proliferation and differentiation.

The present invention also relates to a cell culture kit including the cell culture container of the present invention, and an apparatus for irradiating near-infrared.

Since the kit of the present invention includes the cell culture container using a polymer film as a cell support, proliferation during cell culture is stimulated, cells are detached in an irradiated region by near-infrared, and the polymer film at the cell-detached part is not removed and thus repeatedly used in cell culture and detachment. Particularly, the kit may be effectively used in harvest of stem cells, individual separation of stem cells, or research on a characteristic of one stem cell.

Generally, to detach stem cells in a cell culture container (tissue culture polystyrene) during the harvest of the stem cells, the stem cells being proliferated in the container should be entirely detached using a trypsin enzyme. According to the present invention, when the stem cells are cultured on a surface of a conductive polymer film, the cells may be simply harvested by irradiating near-infrared not harmful to the cells without trypsin, and stem cells having a desired size and in a desired region may be selectively detached. That is, the conventional method is difficult to individually separate stem cells or research a characteristic of an individual stem cell, but the present invention can control a size of detachment region, that is, the number of harvested cells, and individually detach stem cells one by one.

Rays for detaching the cells may be laser beams, and radiation may be performed for 30 seconds to 10 hours at 1 μW/cm² to 300 W/cm², and preferably 100 mW/cm² to 250 W/cm².

Accordingly, the present invention provides a method of detaching cultured cells by irradiating the cell culture container with near-infrared.

In addition, when a conductive polymer film prepared by reducing an oxidized conductive film into a neutral state is used, stem cell proliferation increases compared with that in a common cell culture container, and therefore the conductive polymer film may be useful in cell therapy using stem cells.

Since the stem cells harvested using the conductive film can be normally cultured and proliferated, and when the stem cells are induced to be differentiated into predetermined cells, adult stem cells may be efficiently differentiated into osteocytes, adipocytes, or chondrocytes.

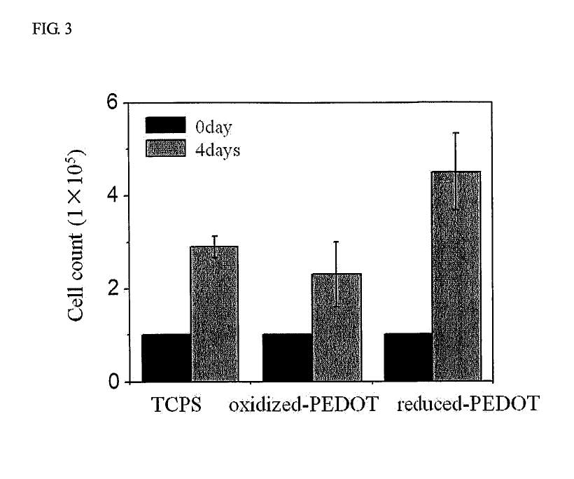

In addition, the conductive film is manufactured and then a doping degree thereof may be controlled as shown in Examples 19 and 20 to be described later. For example, when the doping degree is controlled thereby reducing the conductive film manufactured in an oxidized state into a neutral state as shown in Example 19, as shown as reduced-PEDOT in FIG. 3, cell culture efficiency may be enhanced, compared with the control TCPS used in a conventional cell culture container.

Accordingly, the present invention provides a method of proliferating or differentiating stem cells including culturing adult stem cells in the cell culture container of the present invention.

The present invention also relates to a patterned substrate for cell culture, which includes a substrate, and a cell culture region formed on the substrate and containing a conductive polymer or metal oxide film having an absorbance in a near-infrared region.

The patterned substrate for cell culture is used in culture of cells of blood vessels capable of forming tissues, and may efficiently arrange cells in regularity.

Since the patterned substrate for cell culture uses the film as a cell support, the cells can be detached without enzyme treatment during near-infrared irradiation, and cells may be still cultured in a cell culture region that is not subject to near-infrared irradiation.

The conductive polymer or metal oxide film having an absorbance in the near-infrared region has excellent cell adhesion, and thus cells are possibly attached to a cell culture region without a separate cell adhesive layer.

The substrate may be at least one of insulating substrates such as metal, glass, silicon, or plastic.

The patterned substrate for cell culture may have a patterned non-cell culture region in which a layer inhibiting cell attachment to a cell culture region.

Hereinafter, the present invention will be described in detail by means of Examples. However, it should be understood that the following Example are given by way of illustration of the present invention only, and are not intended to limit the scope of the present invention.

### Preparation Example 1

### Culture of Bone Marrow Mesenchymal Stem Cells

The human bone marrow used herein was normally obtained by consent of a patient approved by Institutional Review Board (IRB) of Severance Hospital, and the experiment was approved by Institutional Review Board (IRB) of Severance Hospital in Korea. Blood obtained from a human bone marrow was subject to Ficoll gradient separation in a ratio of Ficoll-pague:bone marrow blood=1:1.5. A blood sample was slowly poured into a 15 mL Ficoll solution to separate layers, and centrifuged, thereby confirming formation of a thin buffy coat layer on an intermediate layer of a tube, and then the buffy coat layer was separated and transferred to a new tube. Phosphate buffered saline (PBS) was added to the tube to prepare a total 50 ml solution, the solution was centrifuged at 2000 rpm for 10 minutes, a supernatant was discarded, 50 mL PBS was added to a precipitate, the tube was stirred to uniformly mix the contents and then centrifuged again at 1500 rpm for 5 minutes, and a supernatant was discarded, resulting in obtaining cells. The cells were suspended in a medium [DMEM (low glucose)+1% P/S+10% FBS], and then diluted with a medium such that 1×10⁷ cells were included in a 100 mm petri dish (an amount of the medium in the petri dish was designed to 10 mL). After the cells were cultured for one day in a CO₂ incubator, a supernatant was transferred to a new petri dish, and a culture medium having the same components as the medium used in the initial culture was filled on the cells attached to a bottom of the petri dish. After 7 to 10 days, the cells were maintained by being detached using trypsin and seeded in a new flask at 2×10⁵ per T75-flask, resulting in culture and maintenance of adult stem cells.

### Example

### Manufacture of Film Using Conductive Compound

Films were manufactured using conductive polymers of the present invention prepared by polymerizing conductive monomers of Formulas 1a to 1k described above by a method such as solution coating polymerization, vapor polymerization, electropolymerization, or chemical polymerization according to the conditions shown in Table 1. The electropolymerization, vapor polymerization, solution coating polymerization, or emulsion polymerization for preparing particles was used to induce oxidative polymerization of the conductive monomers of the present invention described above, and a polymerization method using a conventionally used catalyst (acid, oxidant, etc.) is a conventional method used in polymerization of a monomer such as a heterocyclic compound or aniline.

To manufacture the conductive polymer film, the conductive polymer could be directly coated on various substrates using the above-described polymerization method. However, a conductive polymer dissolved in a solvent was secondarily coated by spin coating after being synthesized, and conductive polymer particles synthesized by an emulsion method are dispersed in a solvent and then secondarily coated.

In Table 1, a solvent used in electropolymerization is an electrolyte. In addition, when a doping state of the conductive polymer thin film was controlled, the conductive polymer thin film manufactured as described above was put into an electrolyte solution without a monomer, and circulated three times between 1 and −1 V at a rate of 50 mV/s through cyclic voltammetry. At a desired doping voltage (a voltage between 1 to −1 V), the circulation was stopped for several seconds, power was removed, and then a resulting analyte was washed with a pure solvent and dried. In the emulsion polymerization, a value specified at a thickness of the polymer refers to a diameter of a particle. A cell detachment efficiency shown below is a value obtained by converting a ratio of an area of a part from which a cell is detached to an area of a near-infrared radiation region with 100.

TABLE 1

| | | | | near- | near- | |
|---|---|---|---|---|---|---|
| | | Preparation method | Thickness | infrared | infrared | Cell |
| | | & conditions | of | absorbance | radiation | detachment |
| | | (solvent, temperature | polymer | (wavelength: | time | efficiency |
| Example | Compound | (C. °)) | (nm) | 808 nm) | (minutes) | (%) |
| 1 | 1a | Solution coating | 150 | 0.72 | 5 | 100 |
| | | polymerization | | | | |
| | | (butanol, 50) | | | | |
| 2 | 1a | Solution coating | 50 | 0.48 | 10 | 110 |
| | | polymerization | | | | |
| | | (isopropanol, 50) | | | | |
| 3 | 1a | Vapor | 160 | 0.75 | 3 | 100 |
| | | polymerization | | | | |
| | | (isopropanol, 70) | | | | |
| 4 | 1a | Electropolymerization | 250 | 0.88 | 2 | 90 |
| | | (n-Bu₂NClO₄(0.1M)) | | | | |
| 5 | 1b | Solution coating | 130 | 0.65 | 5 | 80 |
| | | polymerization | | | | |
| | | (butanol, | | | | |
| | | isopropanol, 70) | | | | |
| 6 | 1b | Vapor | 150 | 0.7 | 6 | 100 |
| | | polymerization | | | | |
| | | (isopropanol, 80) | | | | |
| 7 | 1c | Solution coating | 150 | 0.68 | 10 | 110 |
| | | polymerization | | | | |
| | | (butanol, 80) | | | | |
| 8 | 1d | Solution coating | 150 | 0.7 | 20 | 110 |
| | | polymerization | | | | |
| | | (butanol, | | | | |
| | | isopropanol, 60) | | | | |
| 9 | 1e | Solution coating | 150 | 0.75 | 30 | 105 |
| | | polymerization | | | | |
| | | (ethanol, 40) | | | | |
| 10 | 1e | Electropolymerization | 140 | 0.7 | 10 | 93 |
| | | (n-Bu₂NClO₄(0.1M)) | | | | |
| 11 | 1f | Solution coating | 350 | 0.9 | 15 | 108 |
| | | polymerization | | | | |
| | | (butanol, 80) | | | | |
| 12 | 1g | Solution coating | 160 | 0.62 | 10 | 90 |
| | | polymerization | | | | |
| | | (butanol, | | | | |
| | | isopropanol, 60) | | | | |
| 13 | 1h | Solution coating | 150 | 0.58 | 10 | 87 |
| | | polymerization | | | | |
| | | (butanol, 90) | | | | |
| 14 | 1i | Solution coating | 500 | 0.85 | 5 | 90 |
| | | polymerization | | | | |
| | | (butanol, | | | | |
| | | isopropanol, 70) | | | | |
| 15 | 1j | Solution coating | 160 | 0.6 | 25 | 89 |
| | | polymerization | | | | |
| | | (butanol, 80) | | | | |
| 16 | Aniline | Solution coating | 250 | 0.78 | 40 | 115 |
| | | polymerization | | | | |
| | | (isopropanol, 50) | | | | |
| 17 | Aniline | Spin coating after | 170 | 0.66 | 5 | 90 |
| | | chemical | | | | |
| | | polymerization | | | | |
| | | (methylene chloride) | | | | |
| 18 | 1k | Emulsion | 150 | 0.72 | 5 | 95 |
| | | polymerization | | | | |
| 19 | 1a | Reduction after | 110 | 0.28 | 30 | 70 |
| | | solution coating | | | | |
| | | polymerization | | | | |
| | | (−0.2 V, 30 sec) | | | | |
| | | (isopropanol, 50) | | | | |
| 20 | 1b | Partial doping after | 120 | 0.55 | 10 | 100 |
| | | vapor polymerization | | | | |
| | | (0.4 V)(isopropanol, | | | | |
| | | 80) | | | | |

### Experimental Example 1

### Near-Infrared Absorbance Test for Film Using Conductive Compound

An absorbance of the conductive polymer film prepared in Example 1 (or 2) was obtained at a range from 200 to 3300 nm using a UV-Visible spectrum. Within the range, the absorbance was shown at 808 nm corresponding to a wavelength of a near-infrared laser in Table 1.

### Experimental Example 2

### Measurement of Photothermal Effect Through Near-Infrared of Film Using Conductive Compound

A conductive polymer film prepared in Example 3 (or 4) was placed on a stand set such that near-infrared was radiated from a bottom thereof, and the photothermal effect was measured. A near-infrared laser at 808 nm was fixed to output energy at 230 mW, and radiated to a bottom of the prepared conductive polymer film. The photothermal effect was confirmed by measuring a temperature of a top of the conductive polymer film through a T-type thermocouple. In the corresponding step, the photothermal effect of the conductive polymer film could be shown as a temperature value measured according to near-infrared laser irradiation time.

As shown in FIG. 2, it was known that the temperature was increased by 25° C. or more by the near-infrared irradiation.

### Experimental Example 3

### Method of Culturing Stem Cells on Film Using Conductive Compound and Selectively Detaching the Stem Cells

The conductive polymer film prepared in Example 8 was sterilized using weak UV rays for approximately 2 minutes, and used as a support in culture of stem cells. Culture of stem cells was performed by putting bone marrow-derived mesenchymal stem cells into a 6-well plate containing a conductive polymer film, and 230 mW of near-infrared was radiated from a bottom of the 6-well plate for selective detachment.

As shown from reduced-PEDOT in FIG. 3, it was noted that, as the stem cells were cultured by using a reduced neutral conductive film as a support, a proliferation rate of the stem cells was higher than that of the stem cells in a common cell culture container, and thus could be effective in cell therapy using the stem cells.

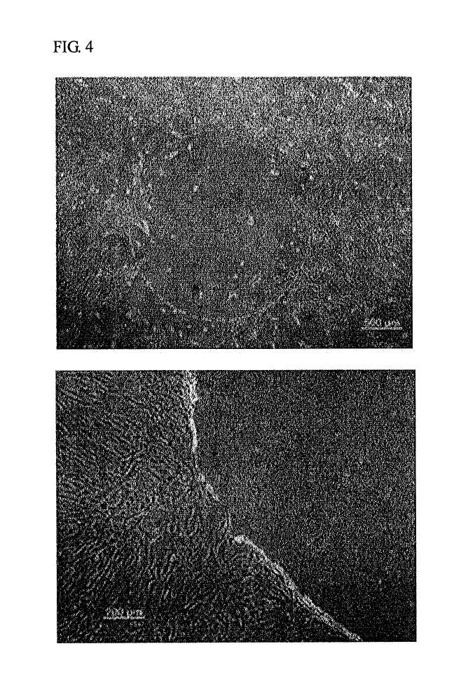

In addition, as shown in FIGS. 4 and 5, a cell detachment area and a cell count were possibly controlled by near-infrared irradiation time.

### Experimental Example 4

### Confirmation of Stem Cell Differentiation

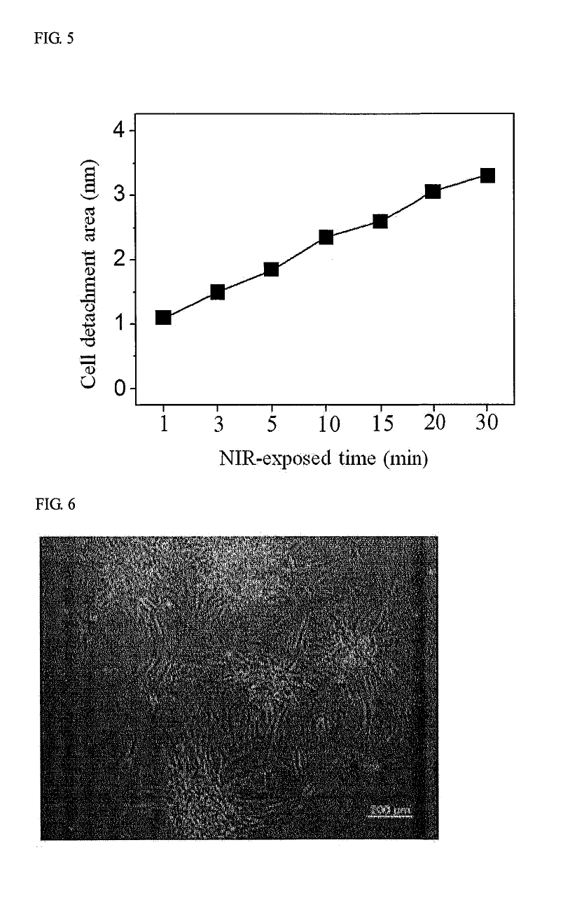

The conductive polymer film prepared in Example 9 (or 10) was sterilized using weak UV rays for approximately 2 minutes, and used as a support in culture of stem cells. Culture of stem cells was performed by putting bone marrow-derived mesenchymal stem cells into a 6-well plate containing a conductive polymer film, and 230 mW of near-infrared was radiated from a bottom of the 6-well plate for selective detachment. FIG. 6 shows various microscope images taken after detached stem cells are transferred to a cell container. Afterward, differentiation was induced for 16 days through conditions for differentiating into osteocytes, adipocytes, and chondrocytes. Here, the group of stem cells cultured on TCPS without a conductive film and then differentiated was determined as a control.

To confirm osteocyte differentiation, after 16 days of the culture, a medium was removed from the control, the cell pellet was washed with PBS, and then the PBS was removed. After the removal, distilled water was added to the cell pellet and then removed, which was repeated three times. A 3% silver nitrate solution filtered through a filter paper was added to the cell pellet, and then stored at room temperature for 30 minutes by covering it with a foil. After 30 minutes, color change in the cell pellet was induced by removing the foil and the added silver nitrate solution and exposing the cell pellet to fluorescent light, and then observed under an optical microscope.

To confirm adipocyte differentiation, after 16 days of the culture, a medium was removed from the control, the cell pellet was washed with PBS, and then the PBS was removed. Here, the cell pellet was treated with 10% formalin, and stayed at room temperature for 30 minutes. Afterward, the formalin was removed, and then the cell pellet was washed with distilled water. After the removal, the cell pellet was treated with 60% isopropanol, and stayed at room temperature for 5 minutes. The isopropanol was removed, and then the cell pellet was treated with oil red-O filtrated through a filter paper and stayed for 10 minutes. After 10 minutes, the cell pellet was washed with tap water until the water became clean, and a degree of dying was observed under an optical microscope.

To confirm chondrocyte differentiation, after 16 days of the culture, a medium was removed from the control, the cell pellet was washed with PBS, and then the PBS was removed. The cell pellet was treated with 1% Safranin-O solution filtrated through a filter paper and stayed for 5 minutes. Afterward, the cell pellet was washed three to four times with 1% acetic acid and then the acid was removed. A degree of dying was observed under an optical microscope.

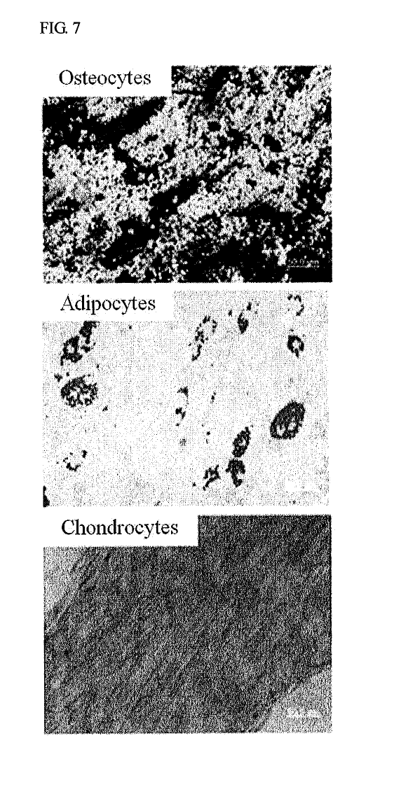

As shown in FIG. 7, it was confirmed that the stem cells detached by near-infrared irradiation were differentiated into osteocytes, adipocytes, or chondrocytes after 16 days like the control.

The present invention is characterized by near-infrared absorption characteristics depending on oxidation and reduction states, and can be used in proliferation, selective detachment, and patterning of cells, particularly, adult stem cells, from a desired location without limitation to time or location using a conductive polymer or metal oxide having a photothermal characteristic during near-infrared irradiation as a support for cell attachment.

The present invention may be used in cell culture.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A method of detaching cultured cells by irradiating a cell culture container with near-infrared, wherein the cell culture container comprises a cell culture region in which a polymer or copolymer film of at least one monomer selected from the group consisting of compounds of Formulas 1a to 1j having an absorbance in a near-infrared region is formed:

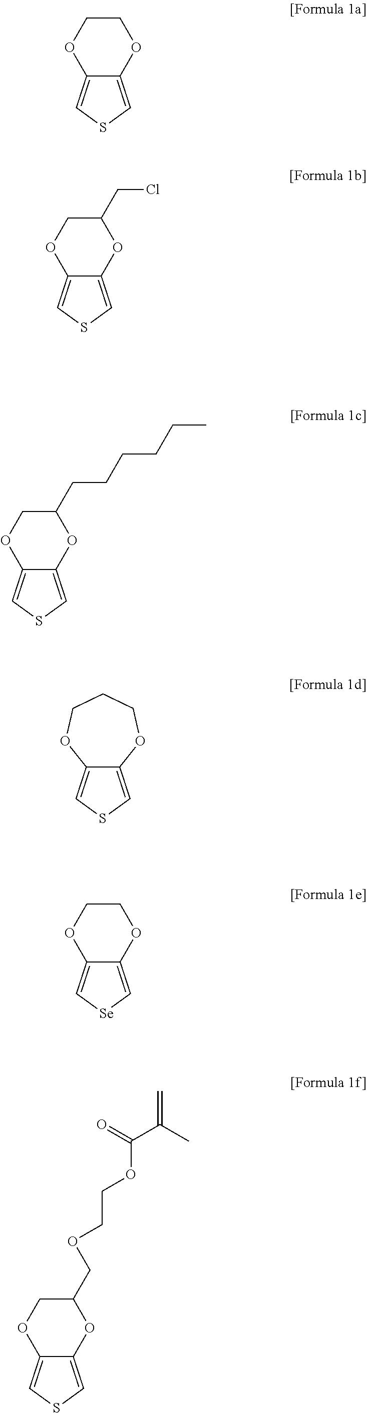 [SMILES: c1scc2c1OCCO2 ; ClCC1COc2cscc2O1 ; CCCCCCC1COc2cscc2O1 ; c1scc2c1OCCCO2 ; c1[se]cc2c1OCCO2 ; C=C(C)C(=O)OCCOCC1COc2cscc2O1]

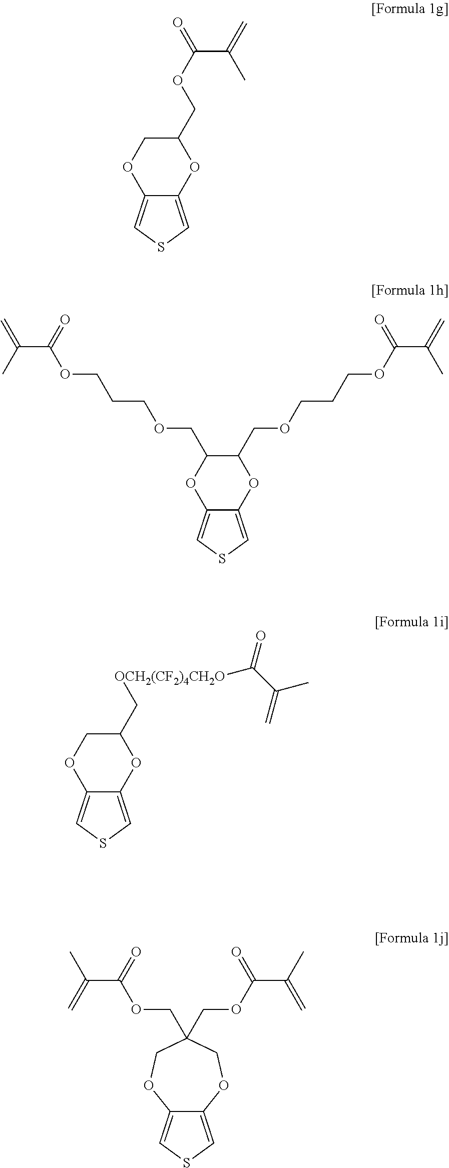

2. The method of detaching cultured cells according to claim 1, wherein the cell culture region is formed of any one of polycarbonate, polypropylene, polyethylene, polystyrene, polyurethane, polyethylene terephthalate, polyester, polyimide, polyethylene glycol, polydimethylsiloxane, and a copolymer or composite thereof; Nylon; paper; cotton; and glass.

3. The method of detaching cultured cells according to claim 1, wherein the polymer or copolymer has a weight average molecular weight of 1,000 to 1,000,000 Da.

4. The method of detaching cultured cells according to claim 1, wherein the polymer or copolymer film has a thickness of 10 nm to 1 mm.

5. The method of detaching cultured cells according to claim 1, wherein the cells include adult stem cells derived from breasts, bone marrow, cord blood, blood, liver, skin, gastrointestine, placenta, or womb.
